(19) United States
(12) Patent Application Publication
(10) Pub. No.: US 2026/0335676 A1
(43) Pub. Date: Oct. 29, 2026

(54) BEAM LIMITING DEVICE, RADIATION DELIVERY SYSTEM, AND RADIATION DELIVERY METHOD

(71) Applicant: SHANGHAI UNITED IMAGING HEALTHCARE CO., LTD., Shanghai (CN)

(72) Inventors: Yanfang LIU, Shanghai (CN); Wei HAN, Shanghai (CN); Fuwei ZHAO, Shanghai (CN); Qiudong WANG, Shanghai (CN); Hua FAN, Shanghai (CN); Can LIAO, Shanghai (CN); Tao CAI, Shanghai (CN); Jian ZHANG, Shanghai (CN); Bin SONG, Shanghai (CN); Jianbin RUAN, Shanghai (CN); Cheng NI, Shanghai (CN)

(21) Appl. No.: 19/433,883

(22) Filed: Dec. 28, 2025

(30) Foreign Application Priority Data
Dec. 25, 2024 (CN) 202411941641.7

(51) Int. Cl. A61N 5/10; G21K 1/02

(52) U.S. Cl. CPC A61N 5/1045; A61N 5/1048; A61N 5/1081; G21K 1/025

(57) ABSTRACT

A beam limiting device, a radiation delivery system, and a radiation delivery method are provided. The beam limiting device includes at least two multi-leaf collimators. The multi-leaf collimators are arranged at different positions in a ray direction. One or more of the at least two multi-leaf collimators are movable along the ray direction.

### CROSS-REFERENCE TO RELATED APPLICATION

This application claims priority to Chinese Patent Application No. 202411941641.7, filed on Dec. 25, 2024, the entire contents of which are hereby incorporated by reference.

### TECHNICAL FIELD

The present disclosure relates to the field of radiation delivery, and in particular, to a beam limiting device, a radiation delivery system, and a radiation delivery method.

### BACKGROUND

Radiation delivery technologies (e.g., radiation therapy, radiation processing, radiation verification, etc.) have been widely applied in various fields. As an example, modern radiation therapy (abbreviated as “radiotherapy”) equipment typically employs multi-leaf collimators to precisely control rays, so as to concentrate the rays on tumors while minimizing damage to healthy tissues. However, conventional equipment often suffers from some limitations. For example, dedicated radiotherapy equipment is only suitable for tumors at specific sites, and collimation accuracy of the dedicated radiotherapy equipment is limited. Conventional radiotherapy equipment, while capable of treating a plurality of sites, is inadequate in precisely controlling the rays, and cannot effectively achieve an optimal radiation delivery effect. Therefore, there is an urgent need for radiotherapy equipment to provide higher ray collimation accuracy and flexibility, so as to achieve an optimal radiation delivery effect under different radiation delivery requirements.

For the issue that radiotherapy equipment fails to satisfy various radiation delivery requirements in the related art, no effective solution has been proposed yet.

### SUMMARY

On this basis, it is necessary to provide a beam limiting device, a radiation delivery system, and a radiation delivery method that can solve the problem that various radiation delivery requirements cannot be satisfied.

In a first aspect, a beam limiting device is provided in an embodiment, including: at least two multi-leaf collimators arranged at different positions along a ray direction. One or more of the at least two multi-leaf collimators are movable along the ray direction.

In some embodiments, each of the at least two multi-leaf collimators is movable along a specified direction and/or rotatable about the ray direction. The specified direction is not parallel to the ray direction.

In some embodiments, the beam limiting device is configured to move based on one or more of the following modes: translation, rotation about a first center inside the beam limiting device, and rotation about a second center outside the beam limiting device.

In some embodiments, each of a first multi-leaf collimator and a second multi-leaf collimator among the at least two multi-leaf collimators includes a leaf group. The leaf group of the first multi-leaf collimator and the leaf group of the second multi-leaf collimator are arranged in a staggered manner.

In some embodiments, motion parameters of a leaf group of each of the at least two multi-leaf collimators are configured to be adjusted according to different radiation delivery regions.

In a second aspect, a radiation delivery system is provided in an embodiment, including: a gantry and a radiation device. The radiation device includes a radiation source and a beam limiting device. The radiation source is configured to output a first ray to a radiation delivery region. The beam limiting device is configured to limit radiation of the first ray and includes at least two multi-leaf collimators arranged at different positions along a ray direction, and one or more of the at least two multi-leaf collimators is movable along the ray direction.

In some embodiments, the radiation delivery system further includes an imaging device. The imaging device includes an imaging source and an imaging detector corresponding to the imaging source. The imaging source is configured to output a second ray to the radiation delivery region. The imaging detector is configured to receive the second ray and determine a detection image of the radiation delivery region.

In some embodiments, the gantry includes an aperture configured to accommodate a radiation delivery target object. The radiation source has one or more of the following move modes: movable along the ray direction, rotatable about an axial direction of the aperture, movable along the axial direction of the aperture, or rotatable about a direction perpendicular to the axial direction of the aperture.

In a third aspect, a radiation delivery method applied to the radiation delivery system described in the second aspect is provided in an embodiment. The method includes: driving each of the at least two multi-leaf collimators in the radiation delivery system to move along the ray direction.

### BRIEF DESCRIPTION OF THE DRAWINGS

FIG. 1 is a schematic diagram of a beam limiting device according to an embodiment.

FIG. 2 is a schematic diagram of a radiation delivery system according to an embodiment.

FIG. 3 is a schematic diagram of a radiation delivery system including an imaging device according to an embodiment.

FIG. 4 is an application environment diagram of a radiation delivery method according to an embodiment.

FIG. 5 is a schematic diagram of a radiation delivery system according to another embodiment.

FIG. 6 is an internal structural diagram of a computer device according to an embodiment.

### DETAILED DESCRIPTION OF THE EMBODIMENT

In order to make the objectives, technical solutions, and advantages of the present disclosure clearer and more comprehensible, the present disclosure will be explained below in further detail with reference to the accompanying drawings and embodiments. It should be understood that the specific embodiments described herein are for the purpose of interpreting the present disclosure only and are not intended to limit the present disclosure.

Unless otherwise defined, technical or scientific terms used in the present disclosure shall have a general meaning as understood by those skilled in the art to which the present disclosure belongs. The terms such as “a”, “an”, “the”, “this”, and “these” in the present disclosure do not denote a quantitative limitation and may be singular or plural. The terms such as “comprise”, “include”, “have”, and any variations thereof used in the present disclosure are intended to cover a non-exclusive inclusion. For example, a process, method, system, product, or device including a series of steps or modules (units) is not limited to listed steps or modules (units) but may include steps or modules (units) not listed or inherent to such process, method, product, or device. The terms such as “connect”, “link”, and “couple” used in the present disclosure are not limited to physical or mechanical connections but may include electrical connections, whether direct or indirect connections. The term “a plurality of” as used in the present disclosure refers to two or more. The term “and/or” describes an associative relationship between associated objects, indicating that three relationships may exist. For example, “A and/or B” may indicate the following three cases: A exists alone, both A and B exist, or B exists alone. Generally, the character “/” indicates that the associated objects are in an “or” relationship. The terms such as “first”, “second”, and “third” used in the present disclosure are merely used to distinguish similar objects and do not denote a specific order of the objects.

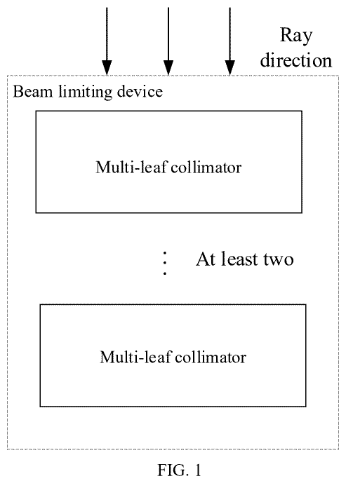

In an embodiment, a beam limiting device is provided in FIG. 1. Referring to FIG. 1, the beam limiting device includes at least two multi-leaf collimators arranged at different positions along a ray direction. One or more of the at least two multi-leaf collimators are movable along the ray direction.

The beam limiting device may be configured to adjust a direction and a shape of a beam of rays output by a radiation source. The radiation source may be a source operable to deliver radiation or irradiation. The rays may include, but be not limited to, one or more of X-rays, gamma rays, protons, heavy ions, and electrons. The radiation source may deliver rays with the same energy or different energies. The rays output by the radiation source may be output to a radiation delivery region after being adjusted by the beam limiting device. The beam limiting device may include the multi-leaf collimators. In an embodiment, the beam limiting device may be the multi-leaf collimators. In an embodiment, in addition to the multi-leaf collimators, the beam limiting device may further include other components, including but not limited to one or more of a fixing mechanism for mounting and fixing the multi-leaf collimators, a housing, a primary collimator, and an auxiliary collimator. The multi-leaf collimators may include a plurality of leaves. Through the multi-leaf collimators, parameters such as the shape of the ray, a resolution of radiation field, and a dosage may be controlled by the leaves in the multi-leaf collimators.

In an embodiment, the multi-leaf collimators may be built into the beam limiting device. For example, a dual-layer multi-leaf collimator may be provided in the beam limiting device. The multi-leaf collimators in the beam limiting device may also be provided in a detachable and/or externally connectable form. For example, when the beam limiting device includes three multi-leaf collimators, two multi-leaf collimators may be built into the beam limiting device, while a third multi-leaf collimator may be provided in an external form. When three multi-leaf collimators are required to jointly adjust the beam of rays output by the radiation source, the third multi-leaf collimator may be connected or moved into the beam limiting device, thereby achieving the effect of incorporating three multi-leaf collimators within the beam limiting device. It may be understood that the number and installation form of the multi-leaf collimators in the beam limiting device may be configured according to actual requirements, which will not be elaborated here.

The multi-leaf collimator may be translated along the ray direction, causing the multi-leaf collimator to approach or move away from a radiation delivery object. In addition, the multi-leaf collimator may be rotated about the ray direction while being translated along the ray direction. To facilitate understanding, taking an initial state of the multi-leaf collimator as being perpendicular to the ray direction as an example, if the multi-leaf collimator is rotated about the ray direction, an included angle between the multi-leaf collimator and the ray direction may be thereby changed.

Alternatively, different multi-leaf collimators may have the same or different movement modes. Specifically, a plurality of multi-leaf collimators may be moved synchronously along the ray direction, or may be moved sequentially, or only a portion of the multi-leaf collimators may be moved. The movement process of the multi-leaf collimators may be continuous or stepwise. The movement mode of the multi-leaf collimators may be configured by a user or determined according to radiation delivery requirements. During the movement of the multi-leaf collimators, the beam limiting device may remain stationary, or the beam limiting device may move together with the multi-leaf collimators.

As a non-limiting example, when the multi-leaf collimators are proximal to the radiation delivery object, a radiation field formed by the rays output by the multi-leaf collimators exhibits increased resolution, a reduced penumbra and a reduced field size. Conversely, when the multi-leaf collimators are moved far away from the radiation delivery object, radiation field formed by the rays output by the multi-leaf collimators exhibits decreased resolution, an increased penumbra, and an increased field size. When the multi-leaf collimators are rotated, a shape of the radiation field formed by the rays output by the multi-leaf collimators may be changed correspondingly. In the present embodiment, by providing the multi-leaf collimators movable along the ray direction, control of the rays and adjustment of parameters of the radiation field can be achieved, thereby satisfying different radiation delivery requirements.

In an embodiment, each multi-leaf collimator is movable along a specified direction that is not parallel to the ray direction, and/or rotatable about the ray direction.

The multi-leaf collimator may move transversely perpendicular to the rays, or may move obliquely.

For example, the specified direction may be a direction perpendicular to the rays. Alternatively, the entire multi-leaf collimator may be translated along the direction perpendicular to the rays, or part of the leaves in the multi-leaf collimator may be controlled to be translated along the direction perpendicular to the rays. When the part of the leaves in the multi-leaf collimator are controlled to be translated, the leaves may be driven to move along a single direction, for example, from a center toward a periphery of the multi-leaf collimator or from the periphery toward the center. Alternatively, different leaves in the multi-leaf collimator may be driven to move along two directions respectively. For example, while a part of the leaves may be driven to move from the center toward the periphery, another part of the leaves may be driven to move from the periphery toward the center. It may be understood that the specified direction may also be a direction not perpendicular to the rays.

The multi-leaf collimator may have a rotation angle determined according to a spatial size and a radiation field range of the beam limiting device. Alternatively, one or more multi-leaf collimators in the beam limiting device may be rotated to a specified angle about the ray direction (e.g., a propagation direction of the rays), thereby adjusting a shape and a size of the radiation field through mutual cooperation between the multi-leaf collimators.

In the present embodiment, the shape of the beam of the rays can be accurately controlled by translating the multi-leaf collimators and/or rotating the multi-leaf collimators about the ray direction, thereby enabling the shape of the field radiation to satisfy different user requirements.

In an embodiment, the beam limiting device may be configured to move based on one or more of the following modes: translation, rotation about a first center inside the beam limiting device, or rotation about a second center outside the beam limiting device.

The beam limiting device may translate along one or more arbitrary directions. Alternatively, the beam limiting device may translate along a horizontal direction, a vertical direction, or other specified directions. Each of the first center and the second center may be a center point or a central axis. The second center may change with a position of the beam limiting device, or may be a fixed position. Alternatively, the first center may be a center point, and the beam limiting device may rotate about this center point. The second center may be a ray axis along which the rays travel, and the beam limiting device may rotate about this ray axis. The beam limiting device may move along with the radiation source, or may move relative to the radiation source. As an exemplary embodiment, the beam limiting device may rotate along with the radiation source about a rotation axis (e.g., an axis of an aperture accommodating a target object to be irradiated) and move along the direction of the rotation axis.

The beam limiting device satisfies angular irradiation requirements by combining various movement modes. Alternatively, irradiation methods of the beam limiting device may include but not be limited to: the beam limiting device rotating about the ray axis (e.g., a central axis of a radiation beam formed by the rays) while moving along a head-foot direction of the radiation delivery object. While rotating about a specific part of the radiation delivery object, the beam limiting device changes the incident angle of the beam of rays by rotating about its own axis.

In this embodiment, by enabling translation of the beam limiting device, the beam limiting device can be moved to the radiation delivery region required for the radiation delivery object. By enabling rotation of the beam limiting device, the beam limiting device can be rotated about a certain part or the entire body of the radiation delivery object. The beam limiting device can achieve not only coplanar irradiation but also non-coplanar irradiation, thereby increasing the degree of freedom in irradiation, and satisfying the angular irradiation requirements.

In an embodiment, each of a first multi-leaf collimator and a second multi-leaf collimator among the multi-leaf collimators may include a leaf group, and the leaf group of the first multi-leaf collimator and the leaf group of the second multi-leaf collimator may be arranged in a staggered manner.

The leaf group may include a plurality of narrow leaves. Alternatively, the leaves in the leaf group may be configured to move independently, or the leaves may move collectively as a unit of a leaf group. When leaf groups are arranged in the staggered manner, the multi-leaf collimator relatively far away from the radiation source can block rays passing through inter-leaf gaps of the multi-leaf collimator relatively proximal to the radiation source. Alternatively, the multi-leaf collimators may constantly maintain a state in which the leaf groups of the multi-leaf collimators are staggered. Alternatively, the multi-leaf collimators may achieve the staggered arrangement of the leaf groups by moving the multi-leaf collimators, when the beam limiting device performs a beam limiting function or when the multi-leaf collimators are moved to a specified position.

In the present embodiment, by arranging the leaf groups of the first multi-leaf collimator and the second multi-leaf collimator in the staggered manner, radiation leakage can be reduced, thereby protecting normal organ tissues adjacent to the radiation delivery region.

In an embodiment, motion parameters of the leaf groups of the multi-leaf collimators may be adjusted according to different radiation delivery regions. The motion parameters may include but not be limited to a leaf position and a moving speed. The motion parameters of the leaf groups of different multi-leaf collimators may be the same or different. Furthermore, for different leaf groups in the same multi-leaf collimator, as well as different leaves in the same leaf group, the motion parameters can be adjusted to different values. The moving of the leaves may include lifting adjustment, horizontal adjustment, rotation, and the like.

Alternatively, a radiation delivery region may be acquired, relationships between current positions of the leaf groups and the radiation delivery region may be determined, and the leaf groups in the one or more collimators may be moved by adjusting the motion parameters. By adjusting the leaf groups, relative positions between different leaf groups can be changed, and inter-leaf gap distances within a leaf group can also be adjusted, so that the region defined by the moved leaf groups may be conformally matched with the radiation delivery region. Alternatively, when the radiation delivery region is relatively large, a relatively wide inter-leaf gap distance may be set by adjusting the motion parameters; when the radiation delivery region is relatively small, a relatively narrow inter-leaf gap distance may be set by adjusting the motion parameters.

Alternatively, the multi-leaf collimators may be respectively associated with different radiation delivery regions. When delivering radiation to a radiation delivery region associated with a certain multi-leaf collimator, the multi-leaf collimator can be used as a primary device for adjusting the rays to adjust a transmission area, a direction and the like of the rays, while other multi-leaf collimators may be used as auxiliary devices of the multi-leaf collimator to perform functions such as blocking the rays for normal organ tissues, cooperatively adjusting the shape and size of the radiation field, and the like.

In the present embodiment, the arrangement and movement manners of the leaves can be changed by adjusting the motion parameters, thereby changing the position and shape of the radiation delivery region and meeting various radiation delivery requirements.

In an embodiment, the leaf groups in different multi-leaf collimators may have different shape parameters. The shape parameters of the leaf group in a multi-leaf collimator may be configured according to a corresponding radiation delivery region. The shape parameters may include but not be limited to one or more of the following parameters: a leaf width, a leaf thickness, an inter-leaf gap distance, and the number of leaves. Alternatively, when the radiation delivery region is relatively large, the associated multi-leaf collimator may be configured with a relatively wide inter-leaf gap distance, a relatively thick leaf width, and a relatively great number of leaves. When the radiation delivery region is relatively small, the associated multi-leaf collimator may be configured with a relatively narrow inter-leaf gap distance, a relatively narrow leaf width, and a relatively few number of leaves. Associatively configuring the multi-leaf collimators according to the radiation delivery regions enables the beam limiting device to be suitable for various radiation delivery requirements.

Based on the same inventive concept, a radiation delivery system for implementing the beam limiting device described above may be provided in an embodiment of the present disclosure. A problem-solving implementation solution provided by the system may be similar to that described in the above device. Therefore, specific limitations in one or more embodiments of the radiation delivery system provided below may be referred to the corresponding limitations for the beam limiting device described above, and detailed descriptions are omitted here.

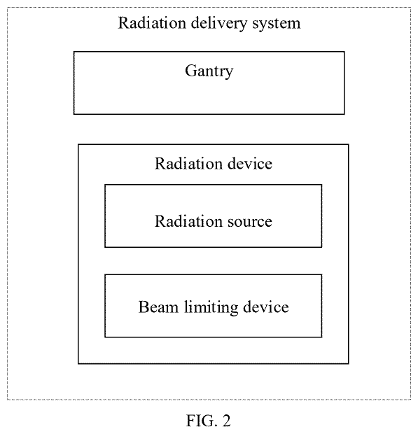

In an embodiment, a radiation delivery system is provided in FIG. 2. Referring to FIG. 2, the radiation delivery system includes a gantry and a radiation device. The radiation device includes a radiation source and a beam limiting device. The radiation source is configured to output a first ray to a radiation delivery region. The beam limiting device is configured to limit radiation of the first ray.

The gantry may be configured to bear or support the radiation device and a radiation device receiving object. Alternatively, the gantry may be one or more of a rotating arm, an auxiliary positioning tool, a treatment couch, a clamping device for a component to be processed, and the like. The radiation source may be a source capable of delivering radiation or irradiation, and the first ray may include X-rays, gamma rays, protons, heavy ions, electrons, or the like. Alternatively, the radiation source may generate X-rays by high-speed electrons bombarding a metal target. The radiation delivery system may achieve functions such as radiotherapy, radiation processing, and phantom dosage verification prior to radiotherapy.

The beam limiting device may be the device described in any of the above device embodiments. In an embodiment, the beam limiting device may include at least two multi-leaf collimators disposed at different positions along the ray direction. One or more of the multi-leaf collimators are movable along the ray direction. The ray direction may include an axial direction of the ray emitted from the radiation device or a direction perpendicular to a plane of an exit portal of the radiation device from which the rays exit. For example, in the case that the ray is cone-shaped, the axial direction may be a central axis direction of the cone. Furthermore, each multi-leaf collimator may be movable along a specified direction and/or rotatable about the ray direction, and the specified direction may be not parallel to the ray direction. Alternatively, each of a first multi-leaf collimator and a second multi-leaf collimator among the multi-leaf collimators may include a leaf group. The leaf group of the first multi-leaf collimator and the leaf group of the second multi-leaf collimator may be arranged in a staggered manner. Alternatively, motion parameters of the leaf group of each multi-leaf collimator may be adjusted according to different radiation delivery regions.

By being arranged to include at least two multi-leaf collimators arranged at different positions along the ray direction, with one or more of the at least two multi-leaf collimators being movable along the ray direction, a broader range of radiation beam adjustment can be achieved compared to moving a single multi-leaf collimator, thereby enabling the radiation beam to satisfy different requirements.

In an embodiment, the beam limiting device may be configured to move based on one or more of the following modes: translation, rotation about a first center inside the beam limiting device, or rotation about a second center outside the beam limiting device.

In an embodiment, the radiation delivery system may further include an imaging device. The imaging device may include an imaging source and an imaging detector corresponding to the imaging source. The imaging source may be configured to output a second ray to the radiation delivery region. The imaging detector may be configured to receive the second ray and determine a detection image of the radiation delivery region. In an embodiment, the imaging device may include a single imaging source. In an embodiment, the imaging device may include a plurality of imaging sources.

The imaging source may be a component in the imaging device that generates or reflects information such as light and electromagnetic waves for imaging. The second ray generated by the imaging source may include, but not be limited to, one or more of the following media: acoustic waves, X-rays, gamma rays, protons, heavy ions, electrons, and the like. The imaging detector may receive the second ray from the imaging source, and may convert the second ray into an electrical signal or other forms of data to obtain the detection image. Alternatively, the imaging detector may be electrically connected to a processor, and the second ray received by the imaging detector may be converted and processed by the processor to obtain the detection image. The imaging detector, disposed corresponding to the imaging source, may be a photoelectric detector, an X-ray detector, an infrared detector, or the like.

The imaging device may be configured to acquire images of the radiation delivery region. Alternatively, the imaging device may be an X imaging device. In this case, the imaging source may be a tube, which utilizes high-speed electrons bombarding a metal target to generate irradiation and output the second ray. The imaging detector may include, but not be limited to, a flat-panel detector or a curved-panel detector. The imaging detector may be, for example, a detector having CT detection and/or DR detection functions. Alternatively, the imaging device may be carried or supported by the gantry. Alternatively, the imaging device may also be a magnetic resonance imaging device. In this case, hydrogen atomic nuclei inside the human body may be used as the second ray output by the imaging source based on the nuclear magnetic resonance principle, eliminating the need for an additional device as the imaging source, but requiring a radio frequency coil to be configured as a detector, with the imaging device provided in a static magnetic field. The imaging device may also be an ultrasonic imaging device, the imaging source may be high-frequency sound waves generated by an ultrasonic generator, and the detector may be an ultrasonic transducer.

The imaging device may determine the detection image at any one or more of the following time points: before the radiation source outputs the ray, during the ray output process, or after the ray is output from the radiation source. Alternatively, taking the imaging source as the tube as an example, during operation of the imaging device, the tube may perform a single exposure or continuous exposures. During the single exposure by the tube, an image may be acquired through one exposure to observe a planar structure of the radiation delivery region, such as a bone structure and positions of internal organs in the radiation delivery region. During continuous exposures by the tube, relatively detailed structural information inside the radiation delivery region, such as structural information of organs and blood vessels, may be acquired through multiple exposures. The single exposure and acquisition of the detection image by the imaging device may enable the user to observe an internal state of the body of the radiation delivery object during the radiation delivery process, while the multiple exposures and acquisition of the detection image by the imaging device may enable the user to inspect the radiation delivery object. In the present embodiment, by providing the imaging device, the user is assisted in monitoring the status of radiation delivery.

In an embodiment, the imaging device may include a plurality of tubes. The plurality of tubes may be arranged at a side of a propagation path of the first ray. Alternatively, the plurality of tubes may be arranged at two sides of the propagation path of the first ray, respectively.

When the plurality of tubes are arranged at the same side, corresponding detectors need to be arranged at the opposite side. When the plurality of tubes are arranged at the two sides, the detectors need to be respectively arranged at the corresponding sides of the plurality of tubes based on the positions of the plurality of tubes. A plurality of imaging devices including the plurality of tubes and corresponding detectors can perform image detection in modes such as simultaneous acquisition, interleaved acquisition, or sequential acquisition.

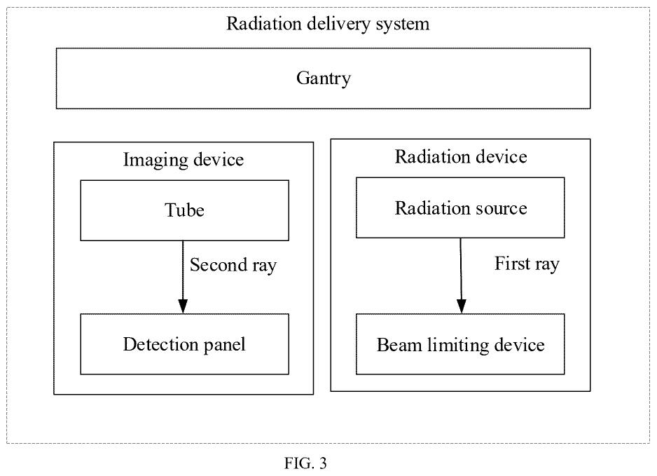

Alternatively, FIG. 3 provides a schematic diagram of a radiation delivery system including an imaging device. Referring to FIG. 3, when a detector is disposed in a form of a detection panel, if there are two tubes and the two tubes are arranged at the same side, a relatively large detection panel needs to be provided. If a plurality of tubes are not arranged at the same side, two detection panels need to be provided simultaneously, but a volume of each of the detection panels is relatively small. Specifically, the positions of the plurality of tubes can be determined according to the actual available space of the radiation delivery system, so as to achieve the effect of reducing spatial costs.

In an embodiment, the gantry may include an aperture configured to accommodate a radiation delivery target object. The radiation source may have one or more of the following move modes: movable along the ray direction, rotatable about an axial direction of the aperture, movable along the axial direction of the aperture, and/or rotatable about a direction perpendicular to the axial direction of the aperture.

The radiation delivery target object may enter a space inside the aperture. Alternatively, after the radiation delivery target object enters the aperture, the radiation source outputs the first ray to the radiation delivery region by adjusting a position of one or more of the radiation source, the beam limiting device, and the aperture accommodating the radiation delivery target object. When the gantry further includes a treatment couch, and the radiation delivery target object is positioned on the treatment couch, the treatment couch together with the radiation delivery target object may enter the aperture accommodating the radiation delivery target object.

Alternatively, the gantry may include a fixed gantry and a movable gantry relative to the fixed gantry. The movable gantry may be coupled to the fixed gantry. The radiation source may be carried or supported by the movable gantry, and the position of the radiation source may be changed by moving the movable gantry to perform one or more of the following motion modes: moving along the ray direction, rotating about the axial direction of the aperture, moving along the axial direction of the aperture, and/or rotating about the direction perpendicular to the axial direction of the aperture.

Taking the radiation delivery target object as a patient as an example, when the radiation source moves along the ray direction, the radiation source may move up and down relative to the patient. When the radiation source rotates about or moves along the axial direction of the aperture, the radiation source may rotate about or move along the head-foot direction of the patient. When the radiation source rotates about the direction perpendicular to the axial direction of the aperture, the radiation source may rotate about a certain center point within a range of 360 degrees. It may be understood that the range of movement and/or rotation of the radiation source may be configured and adjusted according to requirements and the size of the space where the radiation delivery system is located. The radiation delivery target object may also be other objects than the human body. Furthermore, the movable gantry may also carry or support the beam limiting device and the imaging device.

Each module in the radiation delivery system may be implemented entirely or partially in software, hardware, or a combination thereof. The modules may be embedded in or independent of a processor of a computer device in a form of hardware, or may be stored in a memory of the computer device in a form of software, to enable the processor to invoke and execute operations corresponding to the respective modules.

Based on the same inventive concept, a radiation delivery method for implementing the radiation delivery system described above is provided in an embodiment of the present disclosure. The problem-solving implementation solution provided by the method is similar to that described in the system described above. Therefore, specific limitations in one or more radiation delivery method embodiments provided below may be referred to the corresponding limitations for the beam limiting device and the radiation delivery system described above, and detailed descriptions are omitted here.

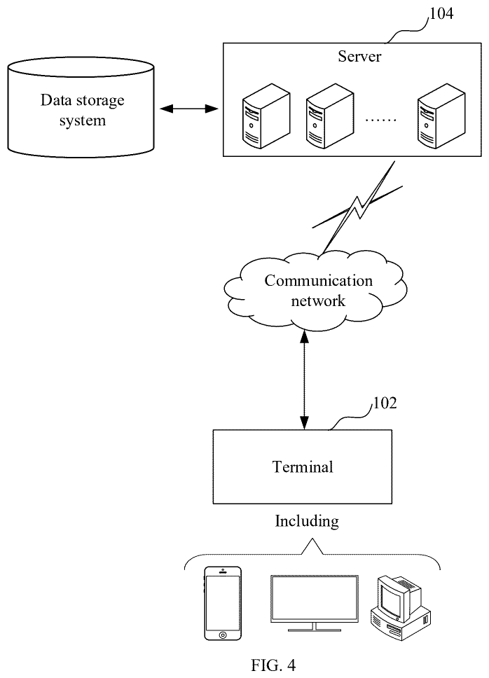

In an embodiment, the radiation delivery method may be applied to the application environment shown in FIG. 4. A terminal 102 may communicate with a server 104 over a network. A data storage system may store data to be processed by the server 104. The data storage system may be integrated into the server 104, or may be located on a cloud or other network servers. The terminal may be electrically connected to the radiation delivery system, and the radiation delivery method may be executed on the radiation delivery system through the terminal. The terminal 102 may be, but not be limited to, various personal computers, laptops, smartphones, tablets, and the like. The server 104 may be implemented as a standalone server or a server cluster including a plurality of servers.

In an embodiment, a radiation delivery method is provided, applied to the radiation delivery system in the above system embodiments. The method is explained as being executed by the terminal in FIG. 4, and includes: driving a multi-leaf collimator in the radiation delivery system to move.

Alternatively, driving one or more multi-leaf collimators in the radiation delivery system to move in the radiation delivery method may include one or more of the following modes: driving the multi-leaf collimators to move along a ray direction; driving the multi-leaf collimators to move along a specified direction non-parallel to the ray direction; or driving the multi-leaf collimators to rotate about the ray direction. The movement of one or more multi-leaf collimators may be driven based on the radiation delivery region and the body position change of the radiation delivery object. A leaf group of each multi-leaf collimator may have motion parameters adjusted according to different radiation delivery regions. A driving device for the multi-leaf collimators may include one or more components including a motor, a position sensor, and the like. Alternatively, each leaf in the multi-leaf collimator may be driven by an independent motor. Alternatively, a preset number of leaves may be grouped together and driven as a unit by an independent motor.

Alternatively, the radiation delivery system may include a first multi-leaf collimator and a second multi-leaf collimator each including a leaf group. The leaf group of the first multi-leaf collimator and the leaf group of the second multi-leaf collimator may remain constantly in a staggered arrangement. Alternatively, based on the radiation delivery method: the first multi-leaf collimator and the second multi-leaf collimator may be driven to move, so that the leaf groups of the dual-layer multi-leaf collimators may be arranged in a staggered manner.

Alternatively, the radiation delivery system may include a first multi-leaf collimator, a second multi-leaf collimator, and a third multi-leaf collimator. The first multi-leaf collimator and the second multi-leaf collimator may be built into a beam limiting device of the radiation delivery system, and the third multi-leaf collimator may either be built into the beam limiting device or externally attached to the beam limiting device. The radiation delivery method may include: when a determination based on radiation delivery requirements indicates that a third multi-leaf collimator is present, driving the third multi-leaf collimator to limit and adjust rays; and when a determination based on radiation delivery requirements indicates that a third multi-leaf collimator is not present, driving the third multi-leaf collimator to move away from the range of the rays.

In this embodiment, by driving the multi-leaf collimators, a shape, a size, a resolution, and a penumbra size of the radiation field can be adjusted, thereby satisfying different radiation delivery requirements.

In an embodiment, the radiation device may include a radiation source and a beam limiting device. The radiation delivery method may further include driving the radiation device to move. The radiation device may be moved based on one or more of the following modes: translation, rotation about a specified center inside the radiation device, and rotation about a specified center outside the radiation device.

Alternatively, the method may further include respectively driving the beam limiting device and the radiation source in the radiation device to move. The beam limiting device may be moved based on one or more of the following modes: translation, rotation about a first center inside the beam limiting device, or rotation about a second center outside the beam limiting device. The radiation source may be moved based on one or more of the following modes: movement along the ray direction, rotation about an axial direction of the aperture, movement along the axial direction of the aperture, and/or rotation about a direction perpendicular to the axial direction of the aperture.

In the present embodiment, by adjusting the radiation device, non-coplanar multi-angle radiation delivery may be achieved, thereby enhancing the radiation delivery effect while reducing damage to healthy tissue.

In an embodiment, the multi-leaf collimators may be respectively associated with different radiation delivery regions, and shape parameters of the leaf groups in the multi-leaf collimators may be configured according to the corresponding radiation delivery regions. The radiation delivery method may further include: determining a target multi-leaf collimator associated with a radiation delivery region, driving the target multi-leaf collimator to adjust a basic shape of a beam of rays, and driving other multi-leaf collimators to further refine the shape of the beam of rays. In the present embodiment, by intelligently selecting the associated multi-leaf collimator for irradiation optimization according to different radiation delivery regions, the radiation delivery effect on the target region can be improved.

In an embodiment, a non-transitory computer-readable storage medium including instructions is provided. The instructions when executed by at least one data processor of a system, cause the system to perform: determining a to-be-treated portion of a radiation delivery target object, determining target positions of at least two multi-leaf collimators along a ray direction according to the to-be-treated portion, and driving the at least two multi-leaf collimators to move to the target positions, respectively.

In an embodiment, the at least two multi-leaf collimators include a first multi-leaf collimator and a second multi-leaf collimator, and the instructions when executed by the at least one data processor of the system, cause the system further to perform: when the to-be-treated portion is a head of the radiation delivery target object, driving the second multi-leaf collimator to move to a first position. The second multi-leaf collimator is more proximal to the head than the first multi-leaf collimator.

In an embodiment, the instructions when executed by the at least one data processor of the system, cause the system further to perform: when the to-be-treated portion is a body of the radiation delivery target object, driving the second multi-leaf collimator to move to a second position. The second position is more proximal to the radiation source than the first position. In an example, a distance between the first position and the head may be equal to or greater than 0.28 meter, and a distance between the second position and the body may be equal to or greater than 0.5 meter.

In an embodiment, the instructions when executed by the at least one data processor of the system, cause the system further to perform: when the second multi-leaf collimator moves to the first position or the second position, adjusting motion parameters of a leaf group of the first multi-leaf collimator or the second multi-leaf collimator, so that the leaf group of the first multi-leaf collimator and the leaf group of the second multi-leaf collimator are arranged in a staggered manner.

In an embodiment, the instructions when executed by the at least one data processor of the system, cause the system further to perform: driving the at least two multi-leaf collimators to move based on one or more of the following modes: translation, rotation about the ray direction.

In an embodiment, the instructions when executed by the at least one data processor of the system, cause the system further to perform: driving a radiation source to move based on one or more of the following modes: moving along the ray direction, rotation about an axial direction of an aperture, moving along the axial direction of the aperture, or rotation about a direction perpendicular to the axial direction of the aperture. The aperture is defined in a gantry and configured to accommodate the radiation delivery target object.

In an embodiment, the at least two multi-leaf collimators are associated with different radiation delivery regions, respectively.

In an embodiment, the instructions when executed by the at least one data processor of the system, cause the system further to perform: determining a target multi-leaf collimator associated with a radiation delivery region among the at least two multi-leaf collimators, driving the target multi-leaf collimator to adjust a basic shape of a beam of rays output from the radiation source, and driving other multi-leaf collimators to further refine the shape of the beam of rays. In an example, the target multi-leaf collimator may adjust the basic shape of the beam of rays output from the radiation source, and the other multi-leaf collimators may block rays passing through the leaf gaps of the target multi-leaf collimator.

In an embodiment, a non-transitory computer-readable storage medium including instructions is provided. The instructions when executed by at least one data processor of a system, cause the system to perform: determining a to-be-treated portion of a radiation delivery target object, and determining a radiation delivery plan according to the to-be-treated portion. The radiation delivery plan includes position parameters of at least two multi-leaf collimators along a ray direction. The position parameters may include parameters of the first position and the second positon of the second multi-leaf collimator and a position of the first multi-leaf collimator.

Specifically, the radiation delivery plan may be determined by plan optimization. When the to-be-treated portion of the radiation delivery target object is the head of the to-be-treated portion of the radiation delivery target object, the second multi-leaf collimator may be configured to adjust a basic shape of a beam of rays output from the radiation source. When the to-be-treated portion of the radiation delivery target object is the body of the to-be-treated portion of the radiation delivery target object, the second multi-leaf collimator may be configured to improve quality of the ream of rays from the first multi-leaf collimator. The second multi-leaf collimator may have an arc shape, and a target point may be focused at a position. When the second multi-leaf collimator is moved to other positions, auxiliary operation may be basically performed by the second multi-leaf collimator, such as blocking rays passing through the leaf gaps of the first multi-leaf collimator.

In an embodiment, the radiation delivery plan may include moving tracks of the at least two multi-leaf collimators. The moving tracks may include at least one of moving along the ray direction, rotation about the ray direction, moving along a direction perpendicular to the ray direction, or leaf moving.

In the related art, radiotherapy equipment mostly employs a single-layer multi-leaf collimator (MLC) structure, which struggles to balance radiation delivery requirements for different parts. A specialized radiotherapy device, such as CyberKnife, is suitable for radiation delivery to tumors in the head or specific parts, but faces technical bottleneck when dealing with tumors in the body. A universal annular radiotherapy device, such as Halcyon, although equipped with a dual-layer MLC structure that provides high leaf resolution through mutual coordination, cannot realize non-coplanar functionality and has certain limitations in treating head tumors. Therefore, neither specialized radiation equipment nor conventional radiation equipment can unify high precision with high adaptability in multi-site tumor radiation delivery.

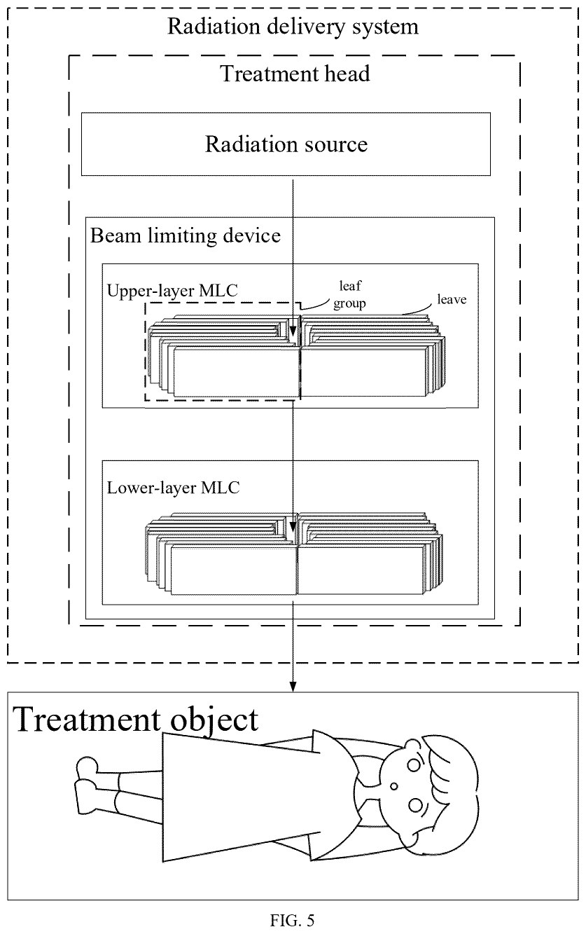

On this basis, in an embodiment, FIG. 5 provides another radiation delivery system for tumor radiation delivery. Referring to FIG. 5, the radiation delivery system may include a treatment head including a radiation source and a beam limiting device, and the beam limiting device may include a dual-layer MLC.

A conventional radiation delivery system can perform irradiation from coplanar angles. To increase the degree of freedom in irradiation, enabling flexible selection of an optimal irradiation angle according to tumor positions when delivering the radiation to complex tumors, a movable treatment head may be provided, allowing the treatment head to rotate within a certain angular range or translate along the head-foot direction of a radiation delivery object, thereby achieving non-coplanar irradiation. Simultaneously, the treatment head may also rotate around the body of the radiation delivery object to achieve coplanar irradiation. The rotation or translation angular range of the treatment head may be determined according to the space available for mounting the radiation delivery system. Specifically, the larger the space for the radiation delivery system, the greater the angular range; the smaller the space for the radiation delivery system, the smaller the angular range.

The MLC in the beam limiting device may include two leaf groups, each including a plurality of leaves. The MLC may or may not include a tungsten jaw. The rays can be output to the radiation delivery region of a treatment object along the gaps between the leaf groups. The cooperation of an upper-layer MLC and a lower-layer MLC may enable optimal radiation delivery for tumors at different sites.

Alternatively, the lower-layer MLC may be configured with the capability to translate vertically along the ray direction. When the lower-layer MLC moves downward, the lower-layer MLC may come more proximal to the radiation delivery object, thereby achieving the advantages of improved resolution and reduced penumbra. The method may be suitable for relatively small tumors. For example, precise radiation delivery to head tumors can be achieved by moving the lower-layer MLC downward. When the lower-layer MLC moves upward, the lower MLC may come more proximal to a metal target of the radiation source. When the lower-layer MLC is moved to a specified range, the cooperation of the upper-layer MLC and the lower-layer MLC may enable the leaf gaps of the upper-layer MLC and the lower-layer MLC to be staggered. In this way, the comprehensive resolution of the leaves may be improved, X-rays passing through the leaf gaps of the upper layer may be blocked, ray leakage may be significantly reduced, adjacent normal organ tissues may be protected, and thus the radiation delivery effect on the tumor region may be improved.

Alternatively, an upper-layer MLC and a lower-layer MLC with independent rotation and cooperative control functions may be provided. The upper MLC layer and the lower MLC layer can independently rotate about an axis where the ray direction is located, enabling seamless integration with a radiation delivery planning system and a real-time tracking system. During the design of the radiation delivery plan, the dual-layer MLCs with coordinated rotation capability can flexibly adjust the shape and size of the radiation field, so that the adjusted radiation field satisfies a radiation field range involved in the radiation delivery planning system. During the radiation delivery process, after real-time postural changes of the radiation delivery object are obtained by combining the real-time tracking system, the dual-layer MLCs may be rotated according to these postural changes to achieve dynamic adjustment of the radiation field, thereby avoiding damage to tissues in peripheral region of the tumor during the radiation delivery process. By configuring the dual-layer MLCs with independent rotation and cooperative control functions, the accuracy of radiation delivery may be improved, and damage to healthy tissues can be reduced.

Alternatively, according to radiation delivery requirements of tumors at different sites, the MLC corresponding to the radiation delivery requirements may be selected for irradiation optimization. Alternatively, when the radiation delivery region is large, such as during radiation delivery to body tumors, the system may prioritize the upper-layer MLC for irradiation optimization to provide a relatively large radiation field, while the lower-layer MLC may cooperate with the upper-layer MLC to assist the upper-layer MLC in optimizing the radiation delivery effect. When the radiation delivery region is small, such as during treatment of head tumors, the system may prioritize the use of the lower-layer MLC for irradiation optimization to provide higher leaf resolution, while the upper-layer MLC may cooperate with the lower-layer MLC to assist the lower-layer MLC in optimizing the radiation delivery effect. An auxiliary MLC may further adjust the radiation delivery parameters within a small range based on the adjustment of the ray by a primarily selected MLC.

Furthermore, the radiation delivery system may further include an imaging device. As an example, the imaging device may include a Digital Radiography (DR) tube and a detector. The DR tube may be used as an imaging source and have the capability of single exposure and/or continuous exposure. The detector corresponding to the DR tube may be either or both of a flat-panel detector and a curved-panel detector. Specifically, a DR tube with single exposure capability combined with the flat-panel detector can achieve DR imaging functions, while a DR tube with continuous exposure capability combined with the curved-panel detector can achieve Computed Tomography (CT) imaging functions. The imaging device may perform image detection before, during, and after radiation delivery of the treatment head. However, the embodiments are not limited thereto, and the imaging device may also include other types, such as a Positron Emission Tomography (PET) detector, a CT tube, a detector, or the like.

The imaging device may include a single DR tube or dual DR tubes. When the imaging device includes the dual DR tubes, the dual DR tubes may be arranged at the same side or two sides of the treatment head along the ray direction. The dual DR tubes, combined with corresponding detectors, form two sets of imaging devices. In terms of acquisition timing, the two sets of imaging devices can realize imaging based on modes such as simultaneous acquisition, interleaved acquisition, or sequential acquisition.

The radiation delivery system for radiotherapy in the present embodiment, by incorporating a treatment head capable of moving freely within a moving range, may have both coplanar and non-coplanar irradiation capabilities. This may enable non-coplanar and multi-angle radiation delivery for complex tumors, enhancing the radiation delivery effect while reducing damage to healthy tissues. The dual-layer MLC structure may provide different combinations of resolution and radiation field range, satisfy the varied radiation delivery requirements of tumors in different regions, be adaptable to tumors in different sites such as the body and the head, and offer high resolution and high adaptability. This may present a novel solution for precise tumor radiotherapy. The MLC capable of moving up and down may significantly improve radiation delivery flexibility, allowing optimal irradiation effects at different distances. The independently rotatable MLCs may enable the upper-layer MLC and the lower-layer MLC to each perform optimal roles in radiation delivery planning and dynamic tracking, achieving higher precision in dosage control. In this way, the applicability of the radiation delivery system for radiotherapy may be improved, the radiation delivery effect of the radiation delivery system for radiotherapy may also be significantly optimized. Meanwhile, significant progress may be achieved in radiation delivery precision and safety, and the defects of related technologies in precise radiation delivery of multi-site tumors may be filled.

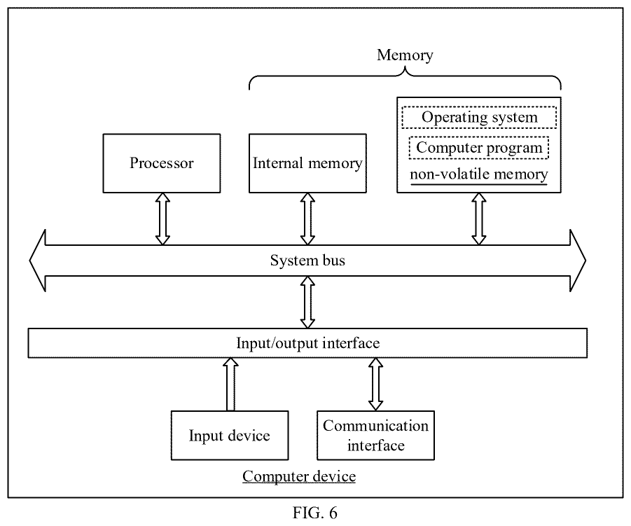

In an embodiment, a computer device is provided. The computer device may be a terminal, which may have an internal structural diagram shown in FIG. 6. The computer device may include a processor, a memory, an input/output interface, a communication interface and an input device. The processor, the memory, and the input/output interface may be connected via a system bus, while the communication interface and the input device may be connected to the system bus through the input/output interface. The processor of the computer device may be configured to provide computing and control capabilities. The memory of the computer device may include a non-volatile storage medium and an internal memory. The non-volatile storage medium may store an operating system and computer programs. The internal memory may provide an environment for operation of the operating system and computer programs in the non-volatile storage medium. The input/output interface of the computer device may be configured to enable information exchange between the processor and external devices. The communication interface of the computer device may be configured to communicate with an external terminal in a wired or wireless manner, and the wireless manner may be implemented via Wi-Fi, mobile cellular networks, near field communication (NFC), or other technologies. The computer programs, when executed by the processor, implement the radiation delivery method. The input device of the computer device may be a touch layer covered on a display screen, or may be a button, a trackball, or a touchpad disposed on a housing of the computer device, or may be an external keyboard, a touchpad, a mouse, or the like.

Those skilled in the art may understand that the structure shown in FIG. 6 is merely a block diagram of partial structures related to the solution of the present disclosure, and does not constitute a limitation on a computer device to which the solution of the present disclosure is applied. A specific computer device may include more or fewer components than those shown in the figure, combine certain some components, or have a different arrangement of components. In an embodiment, a computer device including a memory and a processor may be further provided. The memory stores a computer program, and the processor, when executing the computer program, implements the steps in the method embodiments described above.

In an embodiment, a computer-readable storage medium is provided, having a computer program stored thereon. When executed by a processor, the computer program implements the steps in the method embodiments described above.

In an embodiment, a computer program product is provided, including a computer program. When executed by a processor, the computer program implements the steps in the method embodiments described above.

Those skilled in the art may understand that all or part of the processes in the methods of the above embodiments may be implemented by a computer program instructing relevant hardware. The computer program may be stored in a non-volatile computer-readable storage medium. When the computer program is executed, the method of the above embodiments may be implemented. Any reference to a memory, a database, or other media in the embodiments provided herein may include at least one of a non-volatile memory and a volatile memory. The non-volatile memory may include a read-only memory (ROM), a magnetic tape, a floppy disk, a flash memory, an optical memory, a high-density embedded non-volatile memory, a resistive random access memory (ReRAM), a magneto-resistive random access memory (MRAM), a ferroelectric random access memory (FRAM), a phase change memory (PCM), a graphene memory, and the like. The volatile memory may include a random access memory (RAM), an external cache memory, or the like. By way of illustration and not limitation, the RAM may be in various forms, such as a static random access memory (SRAM) or a dynamic random access memory (DRAM). The database involved in the embodiments provided herein may include at least one of a relational database or a non-relational database. The non-relational database may include a blockchain-based distributed database and the like, and not be limited thereto. The processor involved in the embodiments provided herein may be a general-purpose processor, a central processing unit, a graphics processor, a digital signal processor, a programmable logic, a data processing logic based on quantum computing, or the like, and not be limited thereto.

The technical features in the above embodiments may be combined arbitrarily. In order to make the description concise, not all possible combinations of the technical features in the above embodiments are described. However, as long as there is no contradiction in the combination of the technical features, they should all be considered as falling within the scope of the present disclosure.

The embodiments described above are only illustrative of several implementations of the present disclosure. Although the description is relatively specific and detailed, it should not be construed as limiting the scope of the present disclosure. It should be noted that, for those skilled in the art, several modifications and improvements may be made without departing from the concept of the present disclosure, which all fall within the protection scope of the present disclosure. Therefore, the protection scope of the present disclosure shall be defined by the appended claims.

What is claimed is:

1. A beam limiting device, comprising: at least two multi-leaf collimators arranged at different positions along a ray direction, wherein
one or more of the at least two multi-leaf collimators are movable along the ray direction.

2. The beam limiting device of claim 1, wherein each of the at least two multi-leaf collimators is movable along a specified direction, and the specified direction is not parallel to the ray direction.

3. The beam limiting device of claim 1, wherein each of the at least two multi-leaf collimators is rotatable about the ray direction.

4. The beam limiting device of claim 1, wherein the beam limiting device is configured to move based on one or more of the following modes: translation, rotation about a first center inside the beam limiting device, and rotation about a second center outside the beam limiting device.

5. The beam limiting device of claim 1, wherein each of a first multi-leaf collimator and a second multi-leaf collimator among the at least two multi-leaf collimators comprises a leaf group, and the leaf group of the first multi-leaf collimator and the leaf group of the second multi-leaf collimator are arranged in a staggered manner.

6. The beam limiting device of claim 1, wherein motion parameters of a leaf group of each of the at least two multi-leaf collimators are configured to be adjusted according to different radiation delivery regions.

7. A radiation delivery system, comprising a gantry and a radiation device, wherein the radiation device comprises a radiation source and the beam limiting device of claim 1;
the radiation source is configured to output a first ray to a radiation delivery region; and
the beam limiting device is configured to limit radiation of the first ray.

8. The radiation delivery system of claim 7, further comprising an imaging device, wherein the imaging device comprises an imaging source and an imaging detector corresponding to the imaging source;
the imaging source is configured to output a second ray to the radiation delivery region;
the imaging detector is configured to receive the second ray and determine a detection image of the radiation delivery region.

9. The radiation delivery system of claim 8, wherein the imaging device further comprises a plurality of tubes arranged at a side of a propagation path of the first ray.

10. The radiation delivery system of claim 8, wherein the imaging device further comprises a plurality of tubes arranged at two sides of a propagation path of the first ray, respectively.

11. The radiation delivery system of claim 7, wherein the gantry comprises an aperture configured to accommodate a radiation delivery target object, and the radiation source has one or more of the following move modes: movable along the ray direction, rotatable about an axial direction of the aperture, movable along the axial direction of the aperture, or rotatable about a direction perpendicular to the axial direction of the aperture.

12. A non-transitory computer-readable storage medium comprising instructions, wherein the instructions when executed by at least one data processor of a system, cause the system to perform:
determining a to-be-treated portion of a radiation delivery target object;
determining target positions of at least two multi-leaf collimators along a ray direction according to the to-be-treated portion; and
driving the at least two multi-leaf collimators to move to the target positions, respectively.

13. The non-transitory computer-readable storage medium of claim 12, wherein the at least two multi-leaf collimators comprise a first multi-leaf collimator and a second multi-leaf collimator, and the instructions when executed by the at least one data processor of the system, cause the system further to perform:
when the to-be-treated portion is a head of the radiation delivery target object, driving the second multi-leaf collimator to move to a first position, wherein the second multi-leaf collimator is more proximal to the head than the first multi-leaf collimator.

14. The non-transitory computer-readable storage medium of claim 13, wherein the instructions when executed by at least one data processor of a system, cause the system further to perform:
when the to-be-treated portion is a body of the radiation delivery target object, driving the second multi-leaf collimator to move to a second position, wherein the second position is more proximal to a radiation source than the first position.

15. The non-transitory computer-readable storage medium of claim 14, wherein the instructions when executed by at least one data processor of a system, cause the system further to perform:
when the second multi-leaf collimator moves to the first position or the second position, adjusting motion parameters of a leaf group of the first multi-leaf collimator or the second multi-leaf collimator, so that the leaf group of the first multi-leaf collimator and the leaf group of the second multi-leaf collimator are arranged in a staggered manner.

16. The non-transitory computer-readable storage medium of claim 15, wherein the instructions when executed by at least one data processor of a system, cause the system further to perform:
driving the at least two multi-leaf collimators to move based on one or more of the following modes: translation, rotation about the ray direction.

17. The non-transitory computer-readable storage medium of claim 12, wherein the instructions when executed by at least one data processor of a system, cause the system further to perform:
driving a radiation source to move based on one or more of the following modes: moving along the ray direction, rotation about an axial direction of an aperture, moving along the axial direction of the aperture, or rotation about a direction perpendicular to the axial direction of the aperture, wherein the aperture is defined in a gantry and configured to accommodate the radiation delivery target object.

18. The non-transitory computer-readable storage medium of claim 15, wherein the at least two multi-leaf collimators are associated with different radiation delivery regions, respectively.

19. The non-transitory computer-readable storage medium of claim 18, wherein the instructions when executed by at least one data processor of a system, cause the system further to perform:
determining a target multi-leaf collimator associated with a radiation delivery region among the at least two multi-leaf collimators;
driving the target multi-leaf collimator to adjust a basic shape of a beam of rays output from the radiation source; and
driving other multi-leaf collimators to further refine the shape of the beam of rays.

20. A non-transitory computer-readable storage medium comprising instructions, wherein the instructions when executed by at least one data processor of a system, cause the system to perform:
determining a to-be-treated portion of a radiation delivery target object; and
determining a radiation delivery plan according to the to-be-treated portion, wherein the radiation delivery plan comprises position parameters of at least two multi-leaf collimators along a ray direction.
